(19) United States
(12) Patent Application Publication
(10) Pub. No.: US 2003/0176772 A1
(43) Pub. Date: Sep. 18, 2003

(54) Disposable vaginal speculum having light device

(76) Inventor: Yeong Fang Yang, Tainan (TW)

Correspondence Address: CHARLES E. BAXLEY, 5th Floor, 59 John Street, New York, NY, 10038, US

(21) Appl. No.: 10/094,871

(22) Filed: Mar. 12, 2002

(51) Int. Cl. A61B 1/32

(52) U.S. Cl. 600/220

(57) ABSTRACT

A vaginal speculum includes a handle having a fixed duckbill part disposed on top, a frame adjustably secured to the handle and having a rearward extending bolt. Another or a movable duckbill part is pivotally secured to the frame and includes a hand grip having an oblong hole for receiving the bolt, and a lock nut is threaded to the bolt for securing the hand grip to the frame. A light device is secured to the frame or the duckbill parts for lighting an area between the duckbill parts when the duckbill parts are opened from each other.

### BACKGROUND OF THE INVENTION

1. Field of the Invention

The present invention relates to a vaginal speculum, and more particularly to a vaginal speculum made of disposable materials and having light device.

2. Description of the Prior Art

Various kinds of typical vaginal speculums have been developed for checking or examining the vagina, and each includes a pair of openable duckbill parts for engaging into and for opening the vagina. The duckbill parts of the typical vaginal speculums may not be moved or adjusted relative to each other, such that the typical vaginal speculums may not be used for examining various kinds of vagina.

U.S. Pat. No. 4,686,966 to Tsai discloses the other vaginal speculum having a bolt threaded between the duckbill parts for moving or adjusting the duckbill parts toward or away from each other. However, it takes time to rotate the bolts in order to move or adjust the duckbill parts toward or away from each other; i.e., the duckbill parts may not be easily and quickly moved or adjusted toward or away from each other with the threaded bolt.

Furthermore, the typical vaginal speculums have no light devices provided therein, such that the doctors may not easily examine the vagina. The doctors have to use the other hand to hold an additional light device, or the other nurses are required to hold the additional light device for lighting the vagina.

The present invention has arisen to mitigate and/or obviate the afore-described disadvantages of the conventional vaginal speculums.

### SUMMARY OF THE INVENTION

The primary objective of the present invention is to provide a vaginal speculum including a structure for allowing the duckbill parts to be easily and quickly moved or adjusted toward or away from each other.

The other objective of the present invention is to provide a vaginal speculum including a light device for lighting the vagina and for allowing the doctors to easily examine the vagina.

The further objective of the present invention is to provide a vaginal speculum made of plastic material or the like for allowing the speculum to be disposed after use.

In accordance with one aspect of the invention, there is provided a vaginal speculum comprising a handle including an upper portion having a first duckbill part provided thereon, a frame secured to the handle and including a first bolt extended therefrom, a second duckbill part pivotally secured to the frame, and including a hand grip extended therefrom and having an oblong hole formed therein for receiving the first bolt, a lock nut threaded to the first bolt for moving and securing the hand grip to the frame, in order to open and separate the second duckbill part away from the first duckbill part, and means for lighting an area between the first and the second duckbill parts when the first and the second duckbill parts are opened and separated from each other.

The second duckbill part includes a first end having an aperture formed therein, the lighting means includes a light device engaged in the aperture of the second duckbill part. Alternatively, the second duckbill part includes a casing having a chamber formed therein for receiving the light device.

The casing includes an opening formed therein and communicating with the chamber of the casing and facing and directing toward the first duckbill part, for allowing the light device to light toward the first duckbill part.

The frame includes a pair of arms extended upward therefrom, the arms each includes a pin extended therefrom, the second duckbill part includes a pair of orifices formed therein for rotatably receiving the pins and for pivotally securing the second duckbill part to the arms of the frame.

A device is further provided for adjusting the frame relative to the handle and includes an oblong hole formed in the frame, and includes a second bolt extended rearward from the handle and slidably engaged in the oblong hole of the frame, and a lock nut threaded to the second bolt and engaged with the frame for securing the frame to the handle.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is an exploded view of a vaginal speculum in accordance with the present invention;

FIG. 2 is a partial exploded view of the vaginal speculum;

FIG. 3 is a side view of the vaginal speculum; and

FIGS. 4, 5, 6, 7 are side views similar to FIG. 3, illustrating the operation of the vaginal speculum.

### DETAILED DESCRIPTION OF THE PREFERRED EMBODIMENT

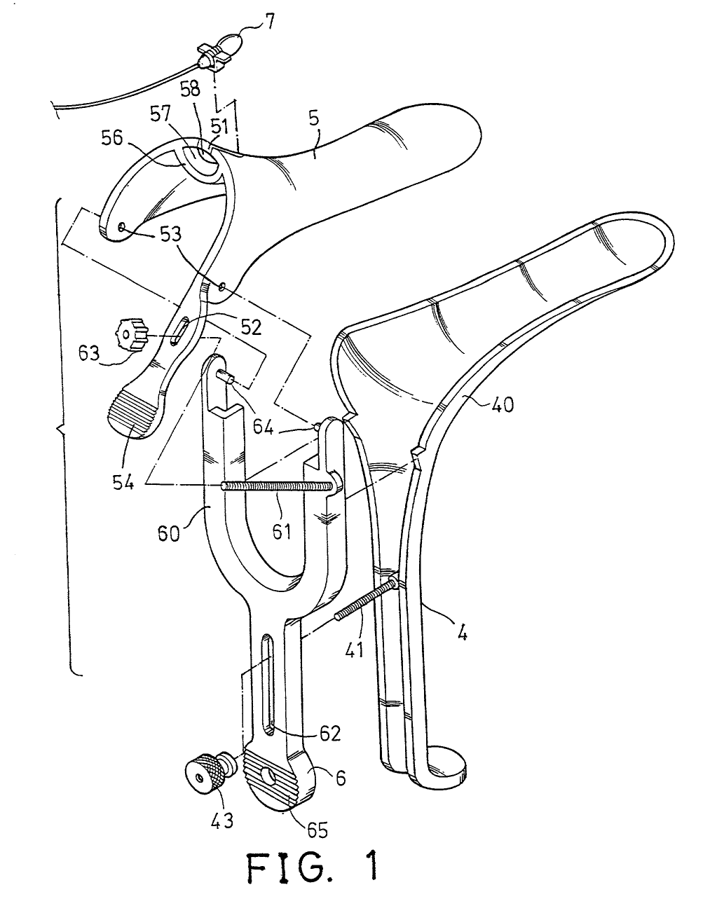

Referring to the drawings, and initially to FIGS. 1-3, a vaginal speculum in accordance with the present invention comprises a handle 4 including a first duckbill part 40 disposed or provided or fixed on top thereof, and preferably perpendicular to the handle 4, but not necessarily be perpendicular to the handle 4. The handle 4 includes a screw or a bolt 41 secured thereto and extended rearward therefrom, such as extended away from the duckbill part 40, for threading with a lock nut 43.

A frame 6 includes an oblong hole 62 formed therein for slidably receiving the bolt 41, and for allowing the frame 6 to be moved and adjusted relative to the handle 4, and for limiting the movement of the frame 6 relative to the handle 4. The bolt 41 may be moved or adjusted along the oblong hole 62 of the frame 6 before the lock nut 43 locks the frame 6 to the handle 4. The frame 6 preferably includes a knurled knob 65 for moving or adjusting the frame 6 relative to the handle 4.

The frame 6 includes a pair of arms 60 extended upward therefrom and spaced away from each other, and preferably parallel to each other, but not necessarily be parallel to each other. One of the arms 60 includes a screw or a bolt 61 secured thereto and extended rearward therefrom, such as extended away from the handle 4 and the duckbill part 40, for threading with a lock nut 63. The arms 60 each includes a pin 64 extended therefrom, particularly, the pins 64 are extended or facing toward each other.

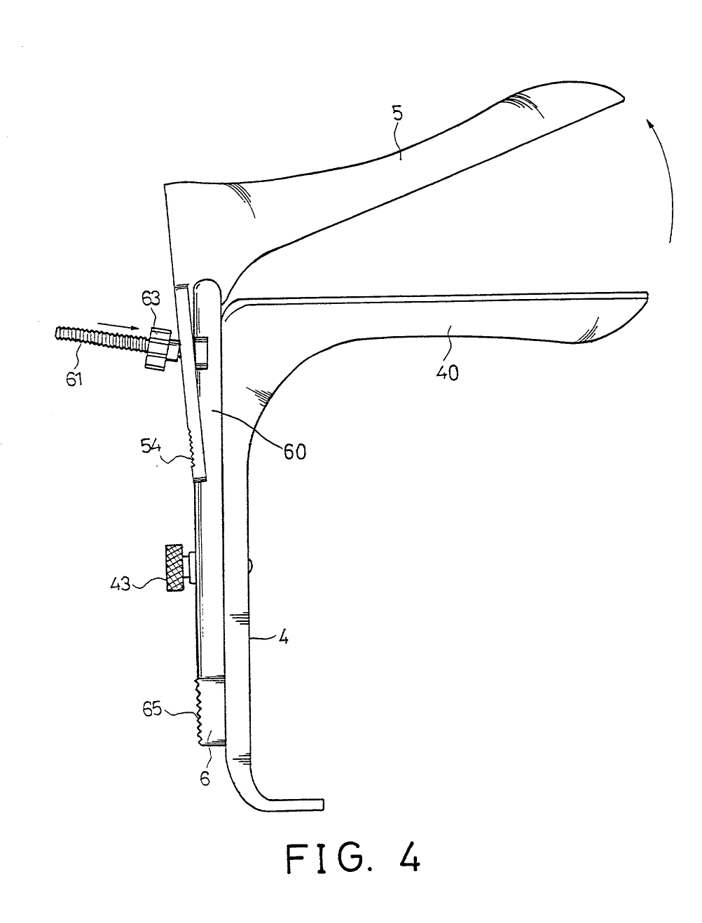

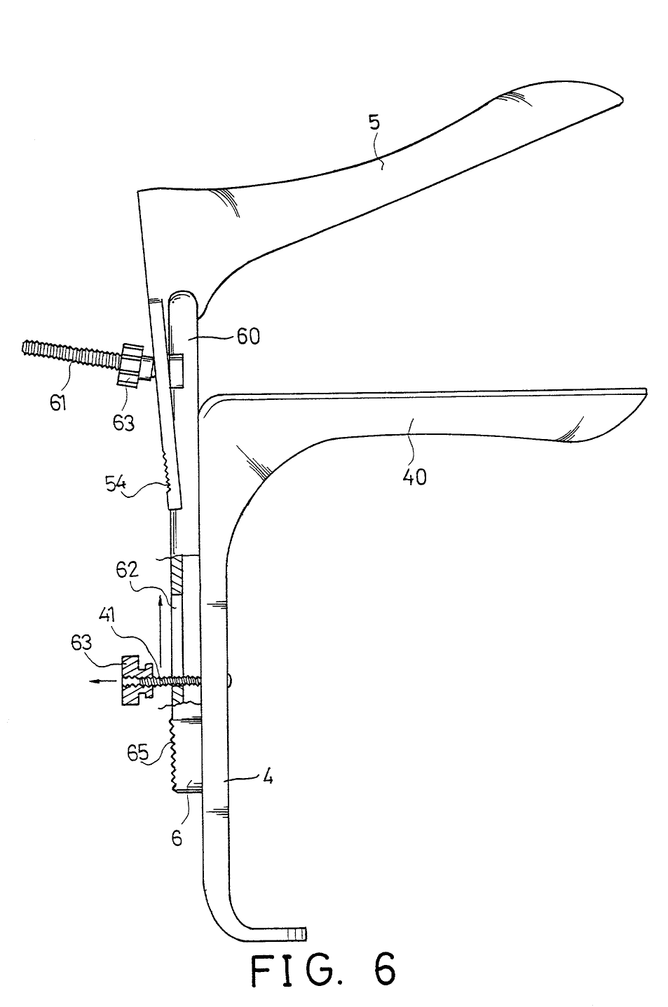

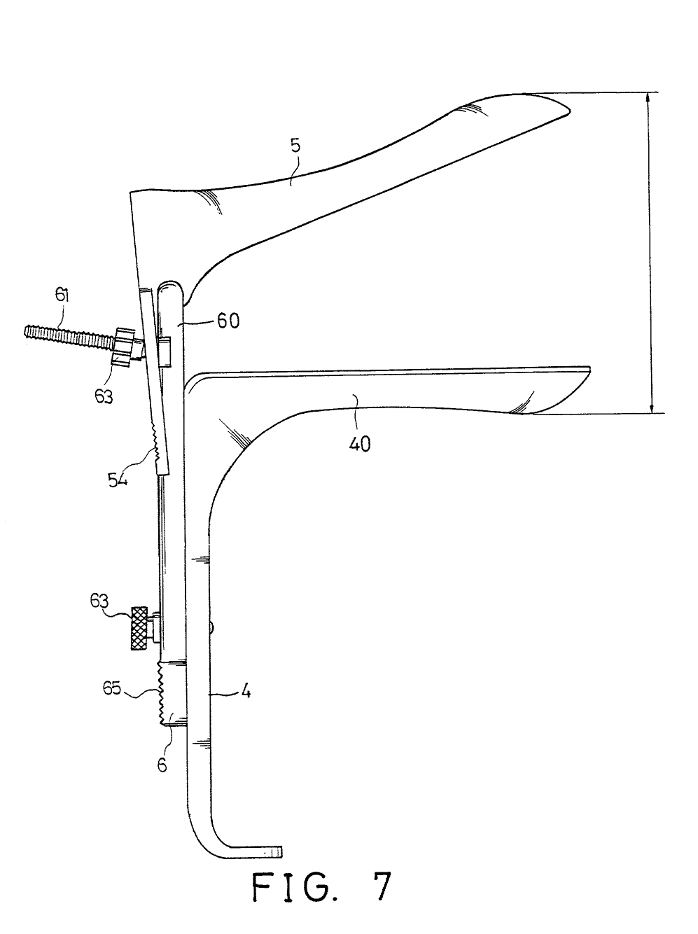

A second duckbill part 5 includes a rear end having a pair of orifices 53 formed therein for rotatably receiving the pins 64 and for allowing the second duckbill part 5 to be rotatably or pivotally secured to the arms 60 of the frame 6. The second duckbill part 5 includes a hand grip 54 extended rearward from one side thereof and having an oblong hole 52 formed therein for slidably receiving the bolt 61, and for allowing the hand grip 54 to be adjusted or moved relative to the bolt 61. The lock nut 63 may lock or secure the hand grip 54 to the arms 60 of the frame 6 (FIGS. 4-7).

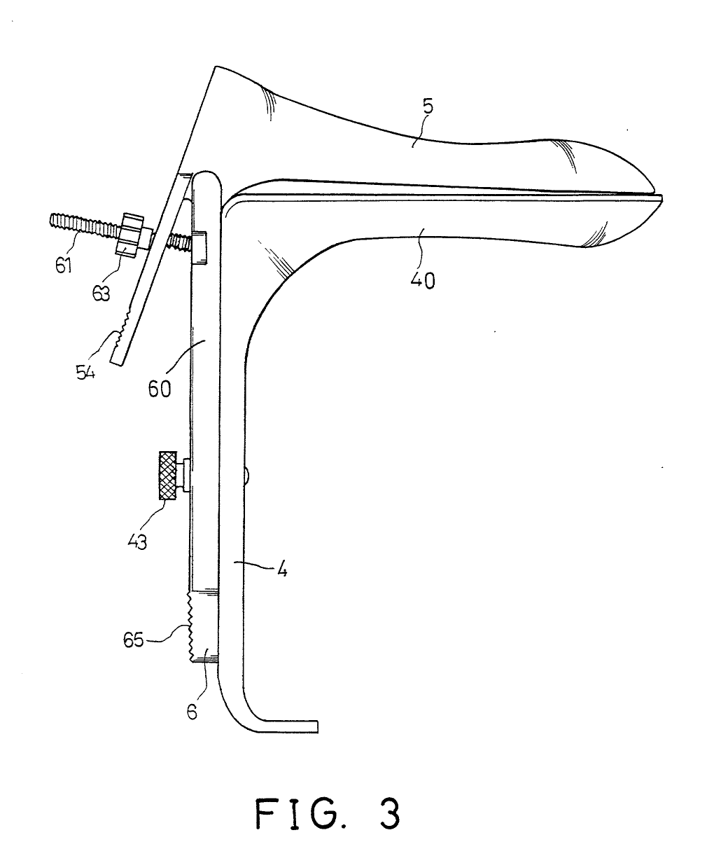

In operation, as shown in FIGS. 3 and 4, the hand grip 54 may be moved toward the arms 60 or the frame 6 to force the hand grip 54 toward or against the arms 60 or the frame 6, in order to open or to move the first and the second duckbill parts 40, 5 away from each other (FIGS. 4-7), and the lock nut 63 may then secure and lock the hand grip 54 to the arms 60 or the frame 6.

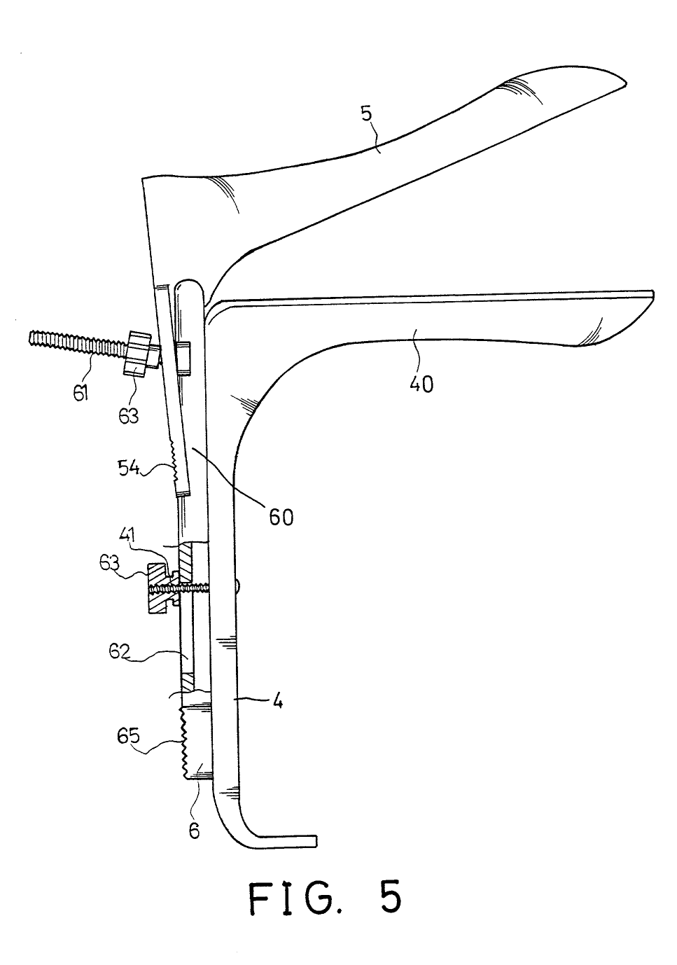

As shown in FIGS. 5-7, the second duckbill part 5 may be moved and adjusted toward or away from or relative to the first duckbill part 40 by moving or adjusting the frame 6 and the arms 60 relative to the handle 4 with the adjustment of the bolt 41 along the oblong hole 62 of the frame 6, such that the duckbill parts 40, 5 of the speculum may be used for engaging into various kinds of vagina.

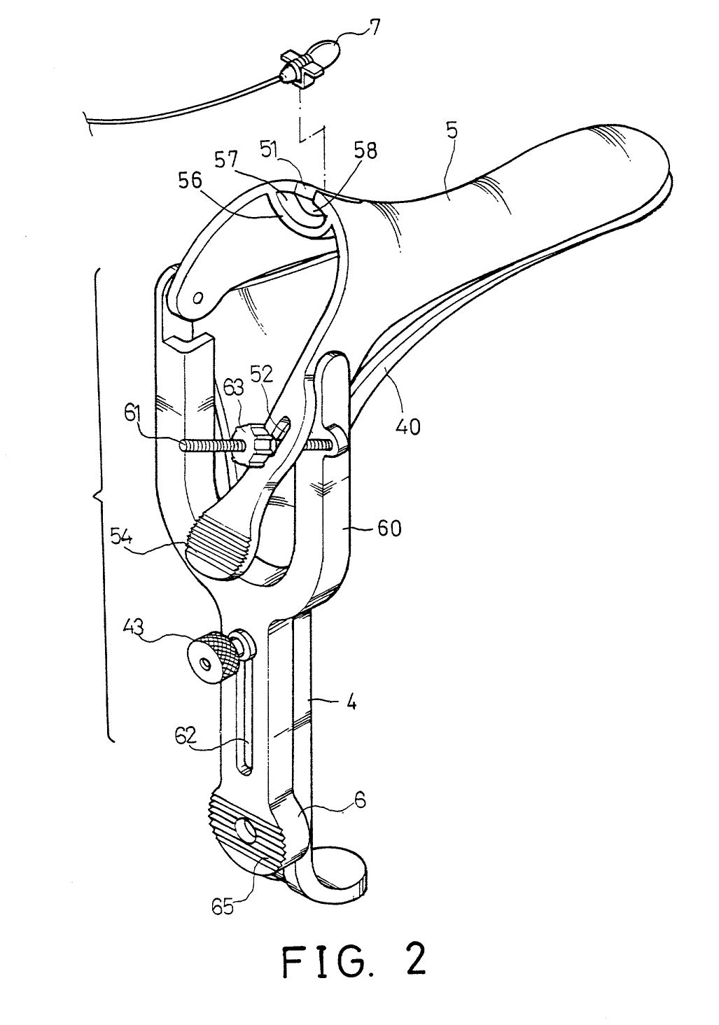

Referring again to FIGS. 1 and 2, the second duckbill part 5 includes a rear portion having an aperture 51 formed therein for receiving a light device 7, and having a casing 56 provided therein for forming or defining a chamber 57 therein and for receiving the light device 7. The casing 56 includes an opening 58 formed therein and communicating with the chamber 57 thereof, and facing or opening inward or toward the first duckbill part 40, for lighting the area between the duckbill parts 40, 5, particularly when the duckbill parts 40, 5 are opened or separated from each other, in order to suitably light the vagina.

Accordingly, the vaginal speculum in accordance with the present invention includes a structure for allowing the duckbill parts to be easily and quickly moved or adjusted toward or away from each other, and/or includes a light device for lighting the vagina and for allowing the doctors to easily examine the vagina, and is preferably made of plastic material or the like for allowing the speculum to be disposed after use.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A vaginal speculum comprising:
a handle including an upper portion having a first duckbill part provided thereon,
a frame secured to said handle and including a first bolt extended therefrom,
a second duckbill part pivotally secured to said frame, and including a hand grip extended therefrom and having an oblong hole formed therein for receiving said first bolt,
a lock nut threaded to said first bolt for moving and securing said hand grip to said frame, in order to open and separate said second duckbill part away from said first duckbill part, and
means for lighting an area between said first and said second duckbill parts when said first and said second duckbill parts are opened and separated from each other.

2. The vaginal speculum according to claim 1, wherein said second duckbill part includes a first end having an aperture formed therein, said lighting means includes a light device engaged in said aperture of said second duckbill part.

3. The vaginal speculum according to claim 1, wherein said second duckbill part includes a first end having a casing formed therein, said casing includes a chamber formed therein, said lighting means includes a light device received in said chamber of said casing of said second duckbill part.

4. The vaginal speculum according to claim 3, wherein said casing includes an opening formed therein and communicating with said chamber of said casing and facing and directing toward said first duckbill part, for allowing said light device to light toward said first duckbill part.

5. The vaginal speculum according to claim 1, wherein said frame includes a pair of arms extended upward therefrom, said arms each includes a pin extended therefrom, said second duckbill part includes a pair of orifices formed therein for rotatably receiving said pins and for pivotally securing said second duckbill part to said arms of said frame.

6. The vaginal speculum according to claim 1 further comprising means for adjusting said frame relative to said handle.

7. The vaginal speculum according to claim 6, wherein said adjusting means includes an oblong hole formed in said frame, and includes a second bolt extended rearward from said handle and slidably engaged in said oblong hole of said frame, and a lock nut threaded to said second bolt and engaged with said frame for securing said frame to said handle.
